(19) United States
(12) Patent Application Publication
(10) Pub. No.: US 2022/0328169 A1
(43) Pub. Date: Oct. 13, 2022

(54) SOFTWARE APPLICATION TO STANDARDIZE ACCESS TO SURGICAL PROCEDURE INVENTORY BENCHMARKS AND DATA COLLECTION AND IMPLEMENTATION OF SURGICAL PROCEDURE BENCHMARKS FOR TRAY RATIONALIZATION AND SUPPLY OPTIMIZATION

(71) Applicants: Benjamin Chadwick Wood, Raleigh, NC (US); David Jon Rowe, Raleigh, NC (US); Martyn Knowles, Raleigh, NC (US)

(72) Inventors: Benjamin Chadwick Wood, Raleigh, NC (US); David Jon Rowe, Raleigh, NC (US); Martyn Knowles, Raleigh, NC (US)

(21) Appl. No.: 17/459,173

(22) Filed: Aug. 27, 2021

Related U.S. Application Data
(60) Provisional application No. 63/172,378, filed on Apr. 8, 2021.

(51) Int. Cl. G16H 40/20

(52) U.S. Cl. CPC G16H 40/20

(57) ABSTRACT

A computerized method that supports the process of data collection for the analysis and rationalization of instrument trays within hospitals, health systems and outpatient surgery includes collecting and storing data on instrument usage on identified high value, volume, and/or frequency instrument trays on a database, and collecting and storing data on preference card instrumentation, equipment and disposables on a database.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### FIELD OF INVENTION

The present invention is directed generally to a software application and benchmarks that support the process of collecting data for the analysis and reconfiguration of instrument trays and disposable supplies for specific surgical procedures within hospitals, healthcare systems and outpatient surgery centers.

### BACKGROUND OF THE INVENTION

Surgical instrument trays have a significant number of re-usable instruments that are not used during cases. Reducing instrument tray size reduces time to set up the operating room, clean and assemble trays, breakdown trays, reconfigure trays and sterilize trays. Furthermore, these improvements result in a significant decrease in instrument inventory levels, maintenance and purchasing of instruments not being used.

### PURPOSE OF THE INVENTION

To reduce the number and range of instruments, instrument trays, and disposable supplies provided for use in surgical procedures in operating rooms to reduce operative expenses, inventory levels and hospital operating expense in general.

### EXISTING TECHNOLOGY COMPARISONS

Currently there are no systems or technology products that perform instrument tray rationalization utilizing empiric usage data collected intraoperatively.

### USERS OF THIS INVENTION

Hospitals, ambulatory surgery centers, sterile processing companies, medical device manufacturers, software platforms, consulting firms.

### BENEFITS OF THIS INVENTION FOR USERS

The following benefits have been observed in the use of the invention:
Reduced surgical case duration.
Reduced sterile processing costs.
Reduced purchase of instruments.
Reduced need for repair of instruments not used on trays.
Reduction in inventory of instruments.
Reduction in overall internal supply chain costs.
Reduction in facility operational expense.
Reduction in turnover time between surgical cases.
Reduction in setup time in the operating room.
Improved accuracy of instrument usage resulting in reduction of excess instruments.
Improved hospital inventory planning.

### BRIEF SUMMARY OF THE DRAWINGS

For a more complete understanding of the present disclosure, and for further details and advantages thereof, reference shall be made to the accompanying drawings.

FIG. 1 provides a generalized view of the end-to-end process and the interaction of the component parts supported by the invention from tray instrument data collection to the sustainment capability to support the new tray configurations and prevent tray configuration degradation or regression to prior state. The predictive model and tray configuration engine both pull data from standard web service interfaces to access benchmark data. of the invention.

FIG. 2 provides the workflow for the identification and data collection capability.

FIG. 3 details how the invention supports the data collection analysis and the calculation for buffer addition and tray configuration. Database and machine learning algorithm interface with web service benchmarks for use in calculations.

FIG. 4 describes how the invention executes the machine learning model and instrument recommendation algorithm for the proposed tray configurations. Data services repository is where benchmark data along with customer specific configurations are stored.

FIG. 5 describes the use of the consolidation engine to reduce the number of trays sharing similar instruments and reduce tray instance proliferation.

FIG. 6 describes the use of the Web Services—Cloud API to serve as the interface for the Clinical Benchmark Database.

### BRIEF SUMMARY OF THE INVENTION

A toolset and embedded application that supports the collection of data regarding instrument usage on instrument trays, both internal and vendor-specific for surgical procedures. The usage data is collected by procedure, preference card, surgeon, surgical specialty, hospital location by tray and instrument per case. Once collected, a rationalization engine utilizes the usage data to configure new trays based on usage and existing proposed configurations. The analytics algorithm displays the list of affected preference cards and instrument associations in other trays that can be consolidated or eliminated with newly proposed trays. Data association visualizations and statistical analysis provide visualizations which identify the intersection of the proposed tray with other existing associated trays to build consolidated trays. A consolidation engine identifies opportunities to consolidate trays for hospitals to reduce the overall number of trays in each case. The engine utilizes the associations between the preference cards for all procedures done within a hospital and calculates the impact on instrument inventory for the proposed transfer of instruments to consolidated trays. Preference cards are then updated to create a closed feedback loop regarding instruments listed on the preference cards for procedure trays.

A machine learning buffering algorithm is used to predict tray configuration based on past usage and other approved tray configurations. The algorithm uses benchmark data that has comparable usage for the same surgical procedure that has been rationalized and standardized within the benchmarking engine.

The buffering algorithm is extended to interact with signaling algorithms to provide the capability to maintain optimal configurations based on actual usage and buffering predictions. Buffering depends on the benchmark engine to predict which instrument or supply is commonly needed above a specific usage-based metric.

The creation of audit process configurations is used in the validation and modification of preference cards based on perioperative feedback. The new preference card configurations and recommended changes to preference cards include individual or low velocity peel pack items identified by the rationalization engine to complete the process.

Web service access to the benchmarks allows applications outside of the proprietary collection tool within this patent application. Access is allowed via a secure web portal and is a request process with specific filters that allows interaction with the benchmark data. Pre-defined results based on filter criteria (which include surgical location type, procedure list, surgical volume) that return results within three (3) categories:
1. Procedure specific instrument and supply recommendations with usage and buffers for requested procedures.
2. Procedure mix instrument usage and optimal tray configuration recommendations.
3. Instrument tray inventory projections based on procedure mix, annual volume, sterilization turnaround time.

### DETAILED DESCRIPTION OF THE INVENTION

The Tray Collection Part

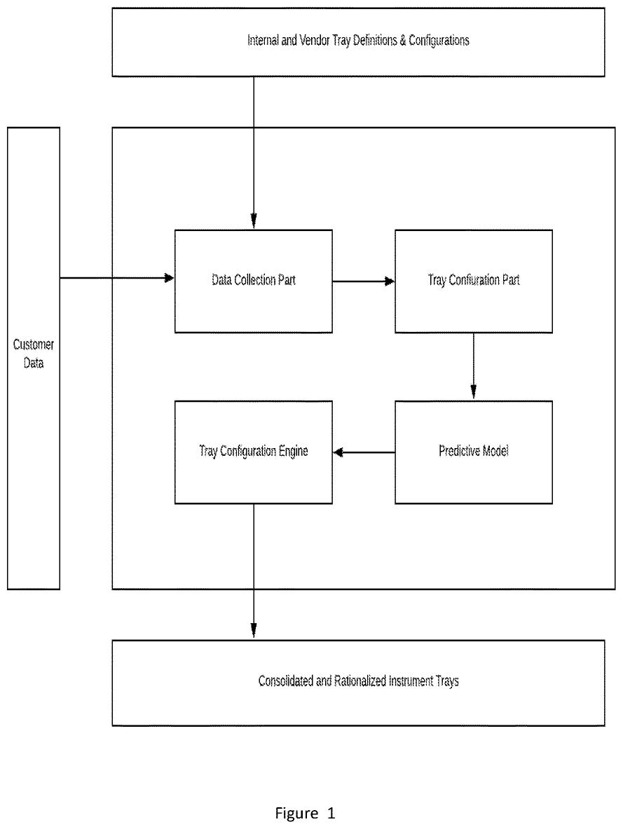

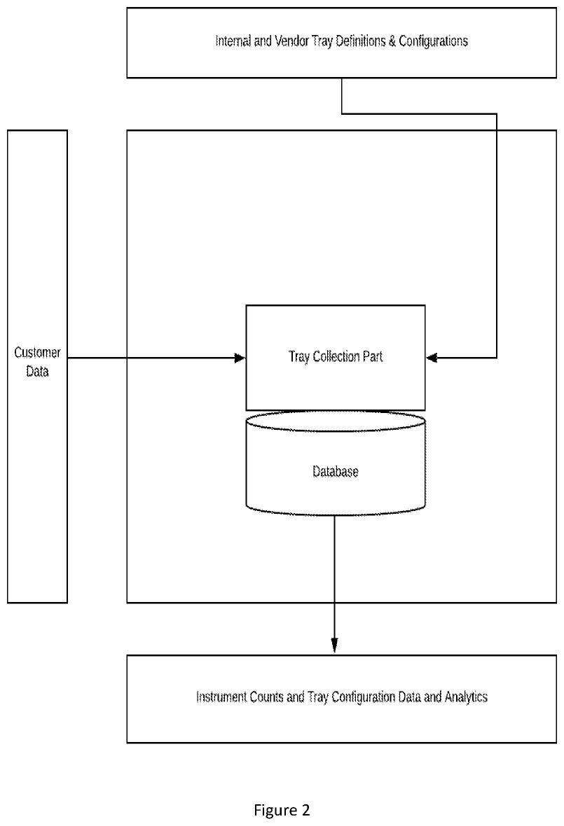

A web application that enables collection of instrument usage during a surgical procedure as detailed in FIG. 1 [0018]. The collection of usage is associated to Instrument Tray, Surgeon, Procedure, Current Procedural Terminology (CPT) code(s), Surgical Specialty, Operating Room, personnel assignments. The workflow for this part of the invention is detailed in FIG. 2 [0019]. The collection process includes an automated notification to signal a team member to count the required trays at the assigned time perioperatively. Quantification of the number of times each procedure has been counted is used to flag cases that need to be counted. Additionally, guidance on cases targeted for data collection is prioritized by Specialty, Location, Surgeon, Procedure and/or CPT code to ensure adequate sample size and count distribution across the aforementioned variables. All data required for collection is integrated into the software to allow collection of counts at a detailed level. Data visibility allows a user of the software to identify data usage for existing trays and create a newly proposed tray configuration. This enables users to see existing tray configurations including the average usage per instrument by Procedure, Surgeon, Location, Preference Card (or Pick List) and the number of counts performed for that tray. Those data allow a user to select commonly used instruments to build a new tray.

The Proposed Tray Configuration Part

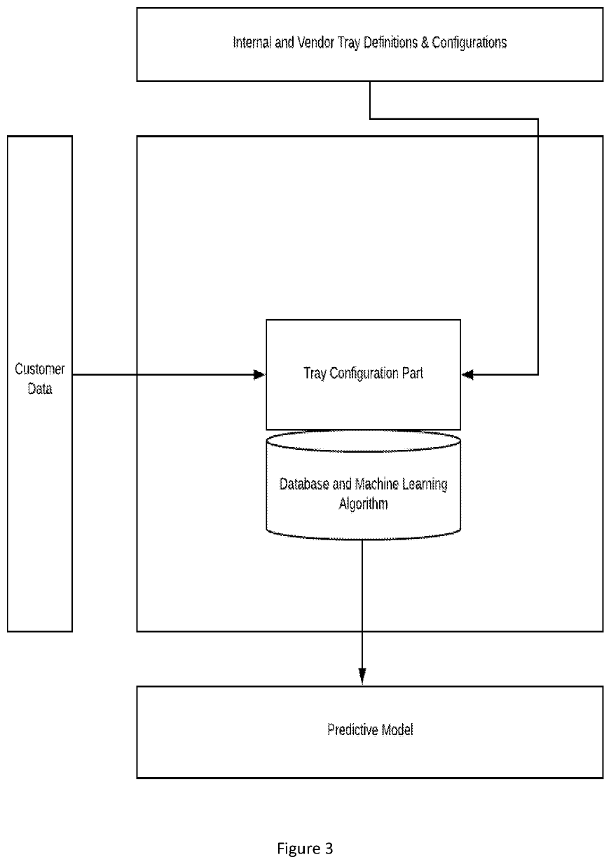

The machine learning algorithm shown in FIG. 3 [0020] generates proposed tray configurations and data-based associations allowing the user to see which existing preference cards or pick lists utilize the proposed tray to understand the scope of the change within the hospital. The tool generates and displays the mathematical percentage of overlap for the newly proposed tray and the existing tray on the preference card or pick list to determine if replacing the tray on the cards is feasible. Iterations of the data can be conducted until a 100% overlap is achieved for a tray associated with the proposed tray allowing the user to replace the existing tray with a new tray. Benchmark data for corresponding tray and supply usage and recommendations are exposed within the tool to allow creation of proposed trays based on benchmarks to reduce amount of sample required.

The Predictive Model

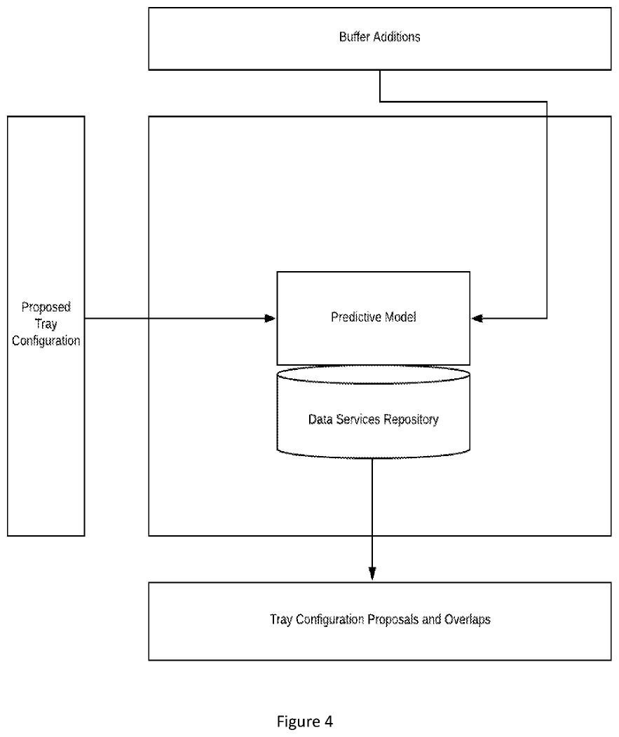

Once the proposed tray is defined and the predictive model shown in FIG. 4 is run, the user will create an audit sheet to be used within the operating room to validate the tray configuration. Surgical staff will pull the newly defined tray configuration instruments from the existing trays on the sterile field at the beginning of the case. At the end of the case, a count will be performed to document the actual usage from the proposed tray. Surgical staff will then provide feedback to add required instruments that are not often used but may be required for patient safety or other reasons. Updates from the audit will be reflected in the proposed tray configurations. After the required number of audits are complete and revisions performed, the new tray is ready for implementation and assignment to preference cards. During the tray rationalization process, a further predictive model utilizing related instruments and tray associations is run to determine based on the Specialty, Location, Surgeon, Procedure and CPT code(s) which instruments are likely needed in addition to the instruments that have actual usage collected in counts. This model identifies specific instruments that would need to be added and the quantity needed.

The model utilizes other approved tray configurations as well as the OpFlow Data Services repository for standardized tray configurations. The model is run and recommendations are made within the application on the tray configuration section. Recommendations must be accepted by the user before they are applied to the tray configuration.

The Tray Consolidation Engine

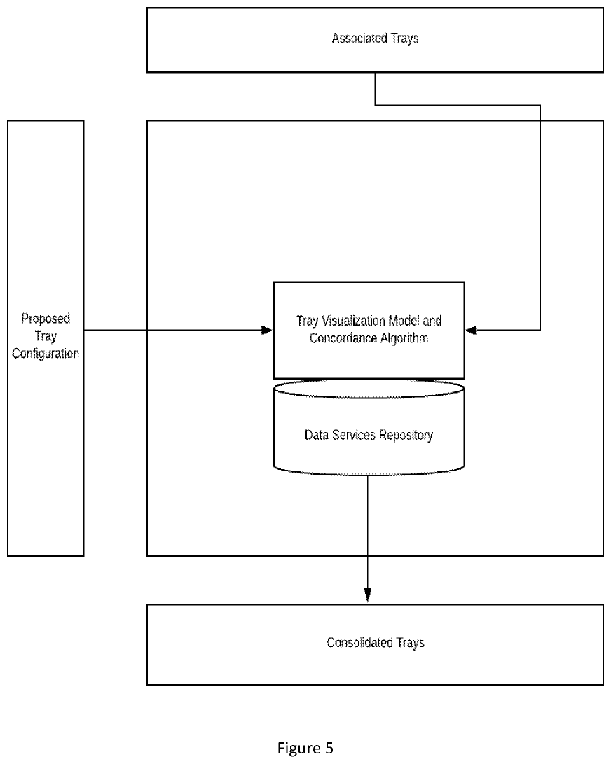

Proposed tray configurations can be used to replace more than one tray that already exists. Within the rationalization process, as depicted in FIG. 1 [0018], the software allows a user to visualize multiple trays in comparison to the proposed trays to see instruments that are on existing trays and allow addition of them to the proposed tray. The visualization allows users to see the concordance of instruments across all trays selected with the proposed tray. Users create a proposed tray configuration that pulls instruments from multiple trays onto one proposed tray by choosing the overlap percentage required for trays associated with the same preference card. The engine then produces the amount of overlap existing between consolidated tray and all the other trays on the associated preference cards as depicted in FIG. 5 [0022]. If 100% coverage exists, then a simple transfer from one tray to another is done. If less than 100% the trays are then presented in descending order of overlap and the highest overlap percentage tray is recommended as the first transfer candidate. Once validated the transfer takes place, the impact on instrument inventory is visible. After the transfer takes place the preference cards are updated creating a closed feedback loop for the new consolidated trays for the relevant procedures.

Synchronization with Preference Cards and Pick Lists

Once complete, the user can view all preference cards that use the existing trays consolidated on the new tray to see if 100% instrument coverage was achieved for cards selected. The software then uses the card assignment process to output a change request for existing preference cards or pick lists to change the existing trays to the newly proposed tray. Users have the ability to pick a specialty, surgeon, location or CPT code(s) to show the instrument coverage for assigning a new tray to existing preference cards. Users can view all preference cards they select and for those cards that have 100% coverage and a case count above their minimum threshold they can select the new tray to replace the existing trays on the cards. A report is generated by the product that shows the Card, Surgeon, Specialty, location and the old tray(s) and new replacement tray. This is used to update external systems that manage the supply chain portions of the surgical process.

Benchmark Web Service Access

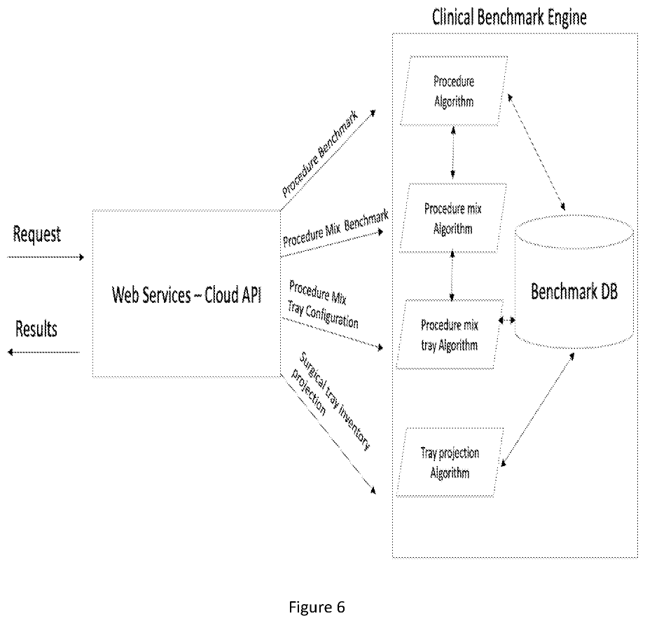

Benchmarks are stored within a database that have the usage, buffer and recommendations based on clinical reviews that can be accessed based on filter criteria exposed via the web services interface as depicted in FIG. 6 [0023]. Authenticated users have four different methods for interacting with the benchmarks that return differing data sets based on each request. Each request allows filter criteria including surgical location type, procedure(s), and service line to limit the data returned from the benchmarks. The interface requests include:
Procedure benchmark by surgical location type that returns average instrument usage, buffer instrument quantity and recommended quantity, average supply usage, supply buffer and supply recommendation;
Procedure mix benchmark by surgical location type and service line that returns average instrument usage, buffer instrument quantity and recommended quantity for multiple procedures requested averaged together;
Procedure mix tray configuration by surgical location type and service line that returns tray name, instrument name, instrument quantity for the trays that are defined within benchmarks for the procedure mix submitted;
Surgical tray inventory projection by surgical location type, service line and procedure mix that returns the project quantity of instrument trays by location that are needed in inventory to support surgical volume for the tray. Request requires historical volume of sterilization by tray per year, time to sterilize each tray, par stock standard by location and number copies of each tray.

The benefits of the benchmark data include:
The determination of what is required for a specific case via being imbedded in other systems;
The ability to assess which procedures have the opportunity to improve based on comparisons and similarities;
A planning process for hospitals based on surgical volume and the needs for trays;
A process to define new tray definitions to streamline configuration.

1-6. (canceled)

7. A computerized method that supports the process of data collection for analysis and rationalization of instrument trays within a healthcare environment, the method comprising using a processor for:
receiving instrument usage information related to instrument trays for use in the healthcare environment;
wherein information related to preference card instrumentation, equipment and disposable articles is stored within a database,
wherein a tray configuration is provided for a determined procedure,
receiving instrument usage information related to instrument trays after use in the healthcare environment;
determining, based on the received instrument usage after use in the healthcare environment, that articles placed within an instrument tray are being used at a frequency below a predetermined threshold;
updating preference card information and tray configuration for the determined procedure based on the determination.

8. The method of claim 7, further comprising receiving information related to surgeons, specialty, and procedure data.

9. The method of claim 7, further comprising determining that some information for a tray configuration is missing for a subsequent procedure, and further comprising generating an alert to alert a user that the subsequent procedure requires data collection.

10. The method of claim 9, further comprising sending the alert to the user.

11. The method of claim 7, further comprising determining a buffer addition for a tray configuration.

12. The method of claim 7, further comprising determining that other tray configurations are related to the updated tray configuration, and updating the other tray configurations based on the updated tray configuration.

13. The method of claim 7, further comprising determining that other tray configurations are associated with the updated preference cards, and updating the other tray configurations based on the updated preference cards.

14. The method of claim 7, wherein the steps of the method are carried out by a machine learning algorithm executing on a computer.

15. The method of claim 7, further comprising reducing a number of instrument trays based on the updated preference cards.

16. The method of claim 7, further comprising causing to display on a remote computer a user interface where queries can be executed by a user, further comprising receiving the queries, and providing search results in response to those queries.
